(19) United States
(12) Patent Application Publication
(10) Pub. No.: US 2015/0250598 A1
(43) Pub. Date: Sep. 10, 2015

(54) ORTHOPEDIC SYSTEM AND METHODS FOR TREATING AN INFECTION

(71) Applicant: Zimmer, Inc., Warsaw, IN (US)

(72) Inventors: Donald L. Yakimicki, Warsaw, IN (US); Verdonna L. Huey, Warsaw, IN (US)

(21) Appl. No.: 14/633,627

(22) Filed: Feb. 27, 2015

Related U.S. Application Data
(60) Provisional application No. 61/947,769, filed on Mar. 4, 2014.

(51) Int. Cl. A61F 2/36; A61F 2/38

(52) U.S. Cl. CPC A61F 2/3609; A61F 2/3859; A61F 2002/3611; A61F 2250/0067; A61F 2310/00395

(57) ABSTRACT

Orthopedic systems and methods for treating an infection are disclosed. An implant can include an exterior shell having an overall shape that defines a shape of the implant and an interior portion. At least a part of the interior portion can be hollow and configured to contain a base material including an antimicrobial or antibiotic. The exterior shell of the implant can include a plurality of perforations formed in at least a part of the exterior shell, and the perforations can release the antimicrobial or antibiotic in the base material from the interior portion of the implant to treat an infection. The base material can include a bone cement, gel, foam or fiber. The implant can be configured for use as a temporary spacer or as a permanent revision implant. After implantation, additional materials can be delivered to the interior portion of the implant and released through the perforations.

### CLAIM OF PRIORITY

This application claims the benefit of U.S. Provisional Patent Application Ser. No. 61/947,769, filed on Mar. 4, 2014, the benefit of priority of which is claimed hereby, and which is incorporated by reference herein in its entirety.

### TECHNICAL FIELD

The present application relates to orthopedic implants, and more particularly, to devices and methods for treating an infection near or around an orthopedic implant.

### BACKGROUND

Orthopedic implants may be used for the replacement of all, or a portion of, a patient's joint. For example, total hip arthroplasty may be used to restore function to a diseased or injured hip joint. Similarly, total knee replacement may be used to restore function to a knee joint.

Once an implant, such as a hip or knee implant, is inside the body, an infection can occur near or around the implant. One or more revision surgeries may be performed to eradicate the infection. In a two-stage revision procedure, after the infected implant is removed, a temporary implant that includes an antibiotic can first be implanted to maintain the joint space, allow the patient some ambulation, and treat the infection. In a second stage, the temporary implant can be replaced with a permanent implant. Thus, this method can require the patient to undergo two separate surgeries as a result of the infection.

In some instances, the temporary implant can include a core material that has an antibiotic bone cement attached to an outside of the core material. This design can require the use of a mold for attaching the bone cement to the core, and such molds are usually not reusable and usually have to be disposed of. Moreover, in these designs, the bone cement commonly serves as the articulating surface.

### OVERVIEW

The present inventors have recognized, among other things, that there is an opportunity for a temporary or permanent implant that can be effectively used to treat an infection without requiring expensive and lengthy processing to make the implant. The implant can be designed such that it can effectively treat the infection and serve as a long term or permanent implant. The implant can be formed such that it can have mechanical strength and improved articulation as compared to existing temporary implants having bone cement as the articulating surface.

To further illustrate the implants and methods disclosed herein, the following non-limiting examples are provided:

In Example 1, an implant can comprise an exterior shell having an overall shape that defines a shape of the implant and an interior portion. At least a part of the interior portion can be hollow and configured to contain a base material including an antimicrobial or antibiotic. The exterior shell can include a plurality of perforations formed in at least a part of the exterior shell. The perforations can be configured to release the antimicrobial or antibiotic in the base material from the interior portion of the implant.

In Example 2, the implant of Example 1 can optionally further comprise a core material located within the interior portion.

In Example 3, the implant of Example 2 can optionally be configured such that the core material is formed of a metal or metal alloy.

In Example 4, the implant of Example 3 can optionally be configured such that the core material is cobalt chrome.

In Example 5, the implant of any one or any combination of Examples 1-4 can optionally be configured such that the exterior shell is formed of a metal or metal alloy.

In Example 6, the implant of Example 5 can optionally be configured such that the exterior shell is formed of a metal or metal alloy.

In Example 7, the implant of any one or any combination of Examples 1-6 can optionally further comprise a feature on the exterior shell or attachable to the exterior shell. The feature can be configured to facilitate injection of the base material into the interior portion of the implant.

In Example 8, the implant of any one or any combination of Examples 1-7 can optionally be configured such that the base material is a bone cement, gel, foam or fiber.

In Example 9, the implant of any one or any combination of Examples 1-8 can optionally be configured such that the implant is configured for use as a permanent revision implant.

In Example 10, a system for treating an infection on or around a joint space can comprise an implant including an exterior shell and an interior portion. The exterior shell can have an overall shape that defines a shape of the implant and has a plurality of perforations formed in at least a part of the exterior shell. At least a part of the interior portion can be hollow. The system can further include a base material including an antibiotic or antimicrobial and a filler device configured for releasable attachment to the implant to deliver the base material into the interior portion of the implant. The interior portion can be configured to contain the base material, and the perforations can be configured to release the antimicrobial or antibiotic in the base material from the interior portion of the implant.

In Example 11, the system of Example 10 can optionally further comprise a core material located within the interior portion of the implant.

In Example 12, the system of Example 11 can optionally be configured such that the base material is releasably contained between an interior surface of the shell and the core material.

In Example 13, the system of any one or any combination of Examples 10-12 can optionally be configured such that the implant includes an articulation surface.

In Example 14, the system of Example 13 can optionally be configured such that the articulation surface is a cap that is inserted over a part of the exterior shell.

In Example 15, the system of any one or any combination of Examples 10-14 can optionally further comprise a film configured to wrap around the implant to secure the implant and immobilize the base material within the interior portion of the implant. The film can be removable prior to inserting the implant inside a body of a patient.

In Example 16, the system of claim 15 can optionally be configured such that the film is a shrink wrap.

In Example 17, the system of any one or any combination of Examples 10-16 can optionally further comprise a port configured for connection to the implant and configured to deliver a second base material into the interior of the implant.

In Example 18, the system of Example 17 can optionally be configured such that the port is a sub-dermal port configured to be implanted into a body of a patient at the same time as the implant.

In Example 19, the system of any one or any combination of Examples 10-18 can optionally be configured such that the implant is a hip implant.

In Example 20, the system of any one or any combination of Examples 10-18 can optionally be configured such that the implant is a knee implant.

In Example 21, the system of any one or any combination of Examples 10-20 can optionally be configured such that the base material is a bone cement, gel, foam or fiber.

In Example 22, a method of making an implant configured for treating an infection on or around a joint space can comprise forming an exterior shell having an overall shape that defines a shape of the implant and forming a plurality of perforations in at least a part of the exterior shell. The method can further comprise injecting a base material into an interior portion of the implant, the base material containing an antibiotic or antimicrobial.

In Example 23, the method of Example 22 can optionally be configured such that forming the exterior shell includes forming the exterior shell around a core such that the interior portion is hollow between an interior surface of the shell and the core.

In Example 24, the method of any one or any combination of Examples 22 or 23 can optionally further comprise attaching a non-perforated cap to the exterior shell, the non-perforated cap configured to be an articulating surface.

In Example 25, the method of any one or any combination of Examples 22-24 can optionally be configured such that the antibiotic or antimicrobial is added to the base material after the base material is injected into the interior portion of the implant.

In Example 26, a method of treating an infection on or around a joint space can comprise providing an implant comprising an exterior shell and an interior portion. The exterior shell can have an overall shape that defines a shape of the implant and have a plurality of perforations formed in at least a part of the exterior shell. At least a part of the interior portion can be hollow. The method can further comprise providing a base material for insertion into the interior portion of the implant. The base material can contain an antibiotic or antimicrobial. The method can further comprise inserting the implant in the joint space such that the perforations in the exterior shell release the antimicrobial or antibiotic in the base material from the interior portion of the implant.

In Example 27, the method of Example 26 can optionally further comprise attaching a port to the implant for delivering additional materials to the interior portion of the implant for release through the perforations in the exterior shell.

In Example 28, the method of any one or any combination of Examples 26 or 27 can optionally be configured such that the additional materials include at least one of irrigation materials, scaffold materials, and osteoinductive agents.

In Example 29, the method of any one or any combination of Examples 26-28 can optionally be configured such that the antibiotic or antimicrobial is added to the base material after the base material is inserted into the interior portion of the implant.

In Example 30, the method of any one or any combination of Examples 26-29 can optionally be configured such that the base material is a bone cement, gel, foam or fiber.

In Example 31, the implant, system or method of any one or any combination of Examples 1-30 can optionally be configured such that all elements or options recited are available to use or select from.

These and other examples and features of the present implants, systems and methods will be set forth in part in the following Detailed Description. This Overview is intended to provide an overview of subject matter of the present patent application. It is not intended to provide an exclusive or exhaustive explanation of the invention. The Detailed Description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a pelvic bone and femur.

FIG. 2 is a perspective view of a hip prosthesis that may be used in a hip replacement surgery.

FIG. 3 is a side view of an example of a hip implant in accordance with the present patent application.

FIG. 3A is a cross-sectional view of the implant of FIG. 3.

FIG. 3B is another example of a cross-sectional view of the implant of FIG. 3.

FIG. 4 is a side view of the implant of FIG. 3 in combination with an example of a filler device in accordance with the present patent application.

FIG. 5 is a side view of the implant of FIG. 3 in combination with another example of a filler device in accordance with the present patent application.

FIG. 6 is a perspective view of an example of a hip implant in accordance with the present patent application.

FIG. 7 is a side view of the implant of FIG. 3 in combination with an example of a sub-dermal port and delivery device in accordance with the present patent application.

FIG. 8 is a perspective view of an example of a knee implant in accordance with the present patent application.

FIG. 9 is a perspective view of an example of a knee implant in accordance with the present patent application.

### DETAILED DESCRIPTION

The present application relates to devices and methods for treating an infection on or around a joint space. After an orthopedic implant has been implanted in the body, an infection can occur over time on or around the implant. This can lead to removal of the implant in order to treat the infection. An implant having a plurality of perforations formed on the exterior shell can replace the original implant and can be configured to release an antibiotic or antimicrobial from an interior of the implant in order to treat the infection. The implant can be a temporary spacer or a permanent revision implant. Additional materials can be injected into the interior of the implant after the implant is implanted in the body.

As used herein, an antibiotic is generally synonymous with an antibacterial, which destroys or suppresses growth or reproduction of bacteria.

As used herein, an antimicrobial is a general term for any compound that can directly act on a micro-organism and can be used for treatment and prevention of infections. Antimicrobials are inclusive of antibacterials, antifungals, and antivirals. An antibiotic is a type of antimicrobial even though the present application may refer to an antibiotic or an antimicrobial.

As used herein, a base material refers to any type of material that can be injected or inserted into an interior portion of an implant and configured to releasably contain an antibiotic or antimicrobial. In an example, the base material can be a flowable material, which refers to a material that is capable of flowing at given conditions and can be injected from one device into another. In some cases the originally-flowable material may no longer be flowable, when exposed to conditions that can change the properties of the material (for example, temperature). An example of this is bone cement, which can initially be in a generally liquid form but can increase in viscosity to where it is no longer injectable as it is curing, ultimately resulting in a non-flowable structure. The flowable material can include one or more materials that are soluble and can be released from the flowable material, including after the flowable material is in a non-flowable state. In an example, the base material can include, but is not limited to, a gel, foam, fiber, woven or non-woven material; although some of these types of materials may not be a ‘flowable’ material, these materials can be injectable from one device to another such that the material can be injected into the interior portion of an implant. In an example, the base material, such as a foam, can be compressible such that the foam can be compressed into a syringe or similar device and then released into the implant, at which point the foam can expand.

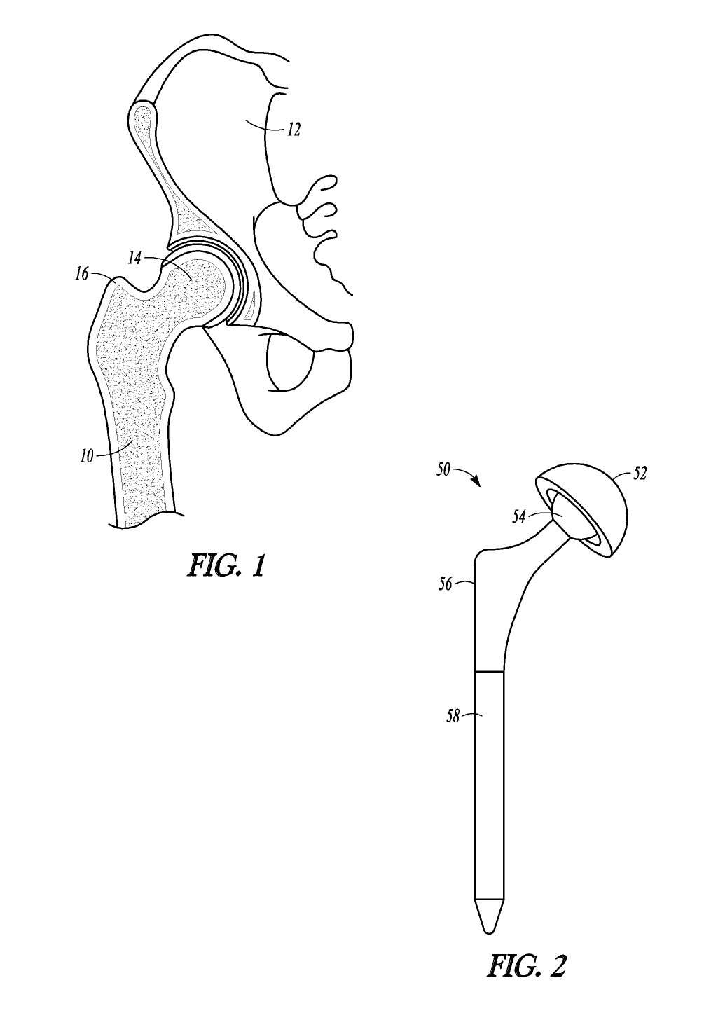

FIG. 1 shows a femur or femoral bone 10 and a pelvic bone 12. As shown in FIG. 1, the femoral bone 10 has different bone regions, including a femoral head 14 and a greater trochanter 16. In hip arthroplasty, at least part of the hip joint is replaced with an implant, such as a prosthesis 50 shown in FIG. 2. The hip prosthesis or implant 50 can include an acetabular shell 52, a femoral head 54, an implant body 56, and a stem 58. In some designs of the prosthesis or implant 50, the implant body 56 can be attached to the stem 58, such as using a nut and threads, taper or other means to engage and connect the implant body 56 to the stem 58. In some designs the implant body 56 and the stem 58 can be a one piece design.

After implantation of the implant 50 in the body of a patient, an infection can develop over time on or around the implant 50. The implant 50 can be removed and replaced with an implant that can treat the infection. Not only does the replacement implant need to treat the infection, but it also needs to maintain joint space and prevent tissue from growing into the space, as well as allow the patient ambulation or generally normal range of motion.

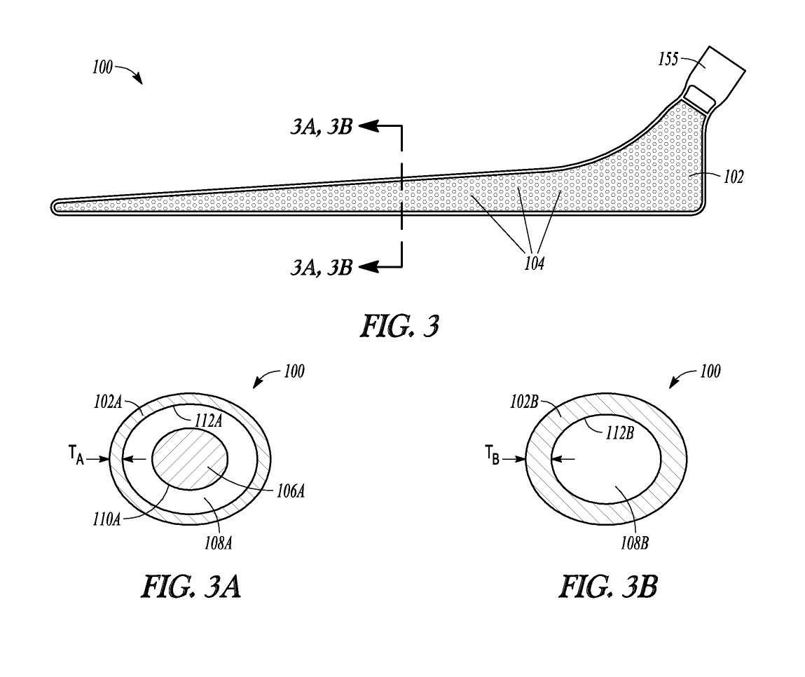

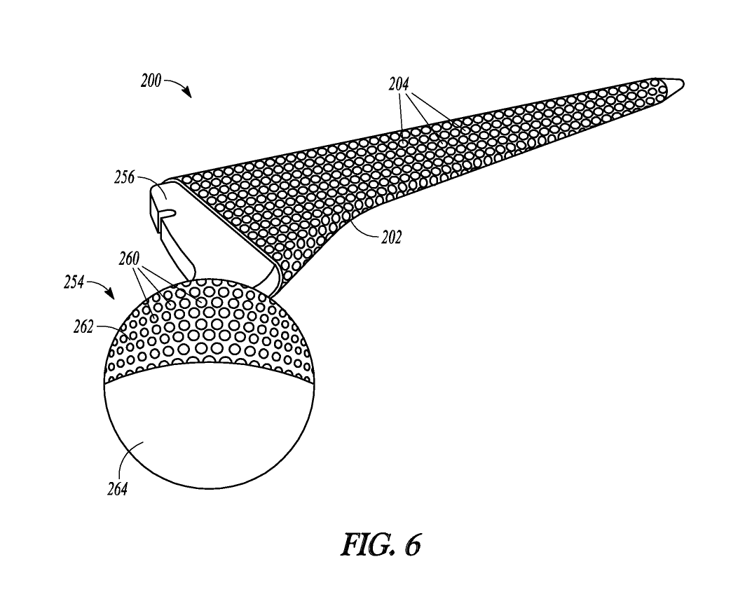

FIG. 3 shows an example of an implant 100, which can be generally similar in size and shape to the implant 50 of FIG. 2, but as shown in FIG. 3, implant 100 does not include a femoral head for engagement with an acetabular shell. A femoral head is described below in reference to FIG. 6. Before implantation, a femoral head can be attached to the implant 100. In an example, an extension 155 of the implant 100 can be received within the femoral head to attach the femoral head to the implant 100. In another example, the femoral head can be integral to the implant and an implant can have a single piece structure including the femoral head. The femoral head may or may not include the perforations described below.

The implant 100 can be a temporary implant or a spacer device used to maintain the joint space after removal of the implant 50, while delivering antibiotics or antimicrobials to treat and eradicate the infection. In that case, the implant 100 can be removed in a subsequent surgery after the infection has been resolved and replaced with a permanent revision implant. Alternatively, the implant 100 can be used as a permanent revision implant that can treat or eradicate the infection, but can also be left inside the body; thus avoiding the need for a second follow-up surgery. The implant 100 can be available in different sizes to accommodate different size hip bones.

The implant 100 can include an exterior shell 102 having an overall shape that defines a shape of the implant. The shell can include a plurality of perforations 104 formed in at least a part of the exterior shell 102. As shown in the example of FIG. 3, essentially all of the implant 100, excluding the femoral head (not shown), has perforations 104 on the exterior shell 102. In other examples, less of an area of the exterior shell 102 than is shown in FIG. 3 can have perforations 104. As described further below, an interior portion of the implant 100 can be filled with a base material, such as an antibiotic bone cement or other material containing an antibiotic or antimicrobial. The base material, or a portion thereof, can be released through the perforations 104 formed in the exterior shell 102. In an example using bone cement, the implant can be filled with the bone cement when the bone cement is in a liquid state and before it hardens. In some cases, a curing process can be used to accelerate hardening of the bone cement. The antibiotic or antimicrobial can be released from the bone cement and then flow through the perforations 104.

In an example, the exterior shell 102 can be formed from bar stock and the perforations 104 can be formed in the exterior shell 102 before or after shaping of the exterior shell 102. In another example, the exterior shell 102 can be formed from a perforated polymeric or metal sheet. In an example, the exterior shell 102 can be made of a polished, perforated sheet metal, such as stainless steel or titanium. In an example, the exterior shell 102 can be made of cobalt chrome. Because the exterior shell 102 can define a shape of the implant 100 and the exterior shell 102 can be formed of a metal or polymeric material, a mold is not needed in order to form the exterior shell 102. Moreover, the exterior shell 102 can provide mechanical strength to the implant 100. The mechanical properties and surface topography of the implant 100 can be controlled based, in part, on the materials and forming processes used.

The exterior shell 102 can be selectively perforated in regions where antiobiotic or antimicrobial elution or release is desired based on the intended placement of the implant 100 inside the body of the patient. In an example, regions of the implant 100 that are configured for articulation, such as femoral heads or knee condyles, can be non-perforated. In an example, components, such as tibial components and hip acetabular components, can have features to attach articulating surfaces, such as liners and cups (for example, ultrahigh molecular weight polyethylene—UHMWPE). Thus the articulating surfaces can have superior articulation. In some examples, the articulating surfaces can also contain antimicrobials.

As stated above, the implant 100 is configured such that a base material, that includes an antimicrobial or antibiotic, can be inserted into an interior portion of the implant 100. The base material, or a portion thereof that includes the antimicrobial or antibiotic, can be later released through the perforations 104 in the exterior shell 102. The types of base materials usable with the implant 100 are described in detail further below. In an example, the interior portion 100 of the implant 100 can also include a core material (see FIG. 3A) and the base material can be contained between the core material and the exterior shell 102. In an example, the core material is excluded from the implant (see FIG. 3B).

FIG. 3A shows an example of an interior portion of the implant 100 of FIG. 3. In an example, the interior portion can include a core material 106A and a hollow portion 108A between an exterior surface 110A of the core material 106A and an interior surface 112A of the exterior shell 102A. The hollow portion 108A can be configured to receive the base material, which can be retained inside the implant 100 until the base material, or a portion thereof including the antibiotic or antimicrobial, is released. The core material 106A can occupy more or less of the hollow portion 108A relative to what is shown in FIG. 3A. The core material 106A can be made of a metal or metal alloy, or other types of material suitable for implantation in the body and having sufficient strength. In an example, the core material 106A can be made of cobalt chrome, titanium or stainless steel. The device can be manufactured by traditional methods such as, for example, metal forming, machining and welding, or by additive manufacturing methods such as, for example, direct laser sintering, selective laser sintering or selective laser melting. By utilizing additive manufacturing technologies, complex geometries such as internal channels and struts can be made possible to optimize the mechanical strength and elution properties of the device. As an example, three-dimensional metal printing can form interior features that may not be achievable using traditional metal forming methods and such interior features can be used, for example, to direct the base material during insertion into the implant 100 or direct the antimicrobial or antibiotic out through the perforations 104. Additive manufacturing technologies can also allow for the manufacturing of patient specific devices to accommodate the patient's anatomy.

FIG. 3B shows an alternative to FIG. 3A of the interior portion of the implant 100. In the example of FIG. 3B, there is no core material and the interior portion includes a hollow portion 108B that can form essentially all of the interior portion (i.e. from an interior surface 112B of the exterior shell 102B). The base material can be retained inside the hollow portion 108B.

The implants of FIGS. 3A and 3B can be designed to have essentially the same or similar strength. Thus, exterior shell 102B can have a greater thickness T_{B} as compared to a thickness T_{A} of the exterior shell 102A of FIG. 3A, since the implant of FIG. 3B does not include a core material. Design of the implant 100 can be optimized for mechanical strength and elution or release of the antibiotic or antimicrobial. In addition to a thickness T of the exterior shell 102, design factors can include, but are not limited to, shape and structure of the core material 106 (if present), material selection for the exterior shell 102, and a size and number of perforations 104 formed in the exterior shell 102.

The implant 100 can be packaged in film, such as shrink wrap or a similar type of material, which can be used as temporary containment to prevent the base material from flowing outside of the implant 100 during filling. (A filling process is described further below.) A shrink wrap or similar type of film can permit the implant 100 to be submerged in water during the exothermic curing of the base material and can prevent thermal degradation of the antiobiotic or antimicrobial included in the base material. By containing the base material, such as bone cement, within the shell 102 and a packaging film, if used, thermal degradation can be reduced or eliminated, as compared, for example, to spacer designs in which the exterior of the spacer is formed of bone cement. As such, different types of antibiotics can be used, for example, in the implant 100 that may otherwise degrade.

In an example, the base material can be inserted into the hollow portion 108 of the implant 100 through a closeable port or a delivery tube or other similar type of device to facilitate insertion of antimicrobial eluting materials, including, but not limited to, porous foams, beads or gels. In an example, the shrink wrap can be used as temporary containment for pressure or vacuum infusion of liquid antimicrobial solutions into porous foam which has been formed inside of or compressed and inserted into the device. In an example, the exterior shell 102 can be treated with an antimicrobial coating such as a silver ion releasing agent.

Because the base material is contained within the interior of the implant 100 when the implant 100 is inserted into the body, other types of materials having an antibiotic or antimicrobial can be used in addition to bone cement. A few examples are provided herein but other types of materials containing antibiotics and antimicrobials, and other types of delivery mechanisms, in addition to what is shown here, can be used with the implant 100.

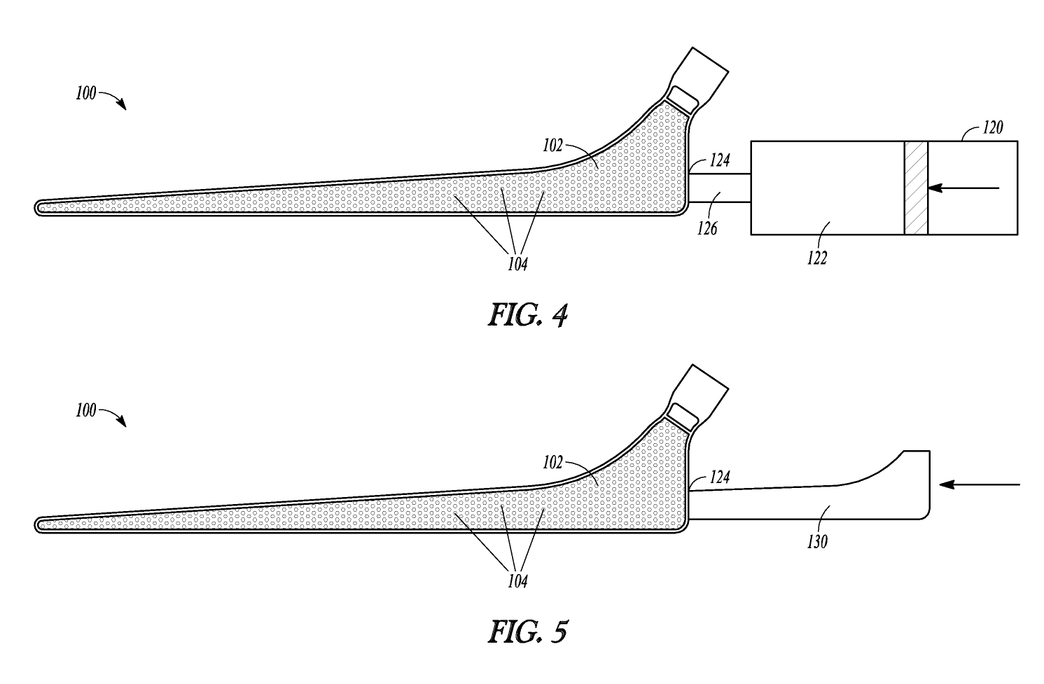

FIG. 4 shows the implant 100 of FIG. 3 and a filler device 120 for injecting a base material 122 into the hollow portion 108 (see FIGS. 3A and 3B) of the implant 100. The exterior shell 102 of the implant 100 can include a feature 124 configured to receive or attach to a feature 126 on the filler device 120 such that the base material 122 flows from an inside of the filler device 120 and into the hollow portion 108 of the implant 100. In an example, the base material 122 is an antibiotic bone cement, and the bone cement is inserted into the hollow portion 108 prior to implantation of the implant 100 in the body of a patient. A curing step can be performed prior to implantation. After the implant 100 is implanted, bodily fluids can flow through the perforations 104 in the exterior shell 102 and into the interior of the implant 100, at which point the body fluids can be exposed to the bone cement. Once the bodily fluids and cement mix with each other, the bodily fluids can dissolve the antibiotic contained within the body cement and the antibiotic can flow out of the interior of the implant 100 through the perforations 104.

In an example, the bone cement can be inserted into the implant as part of the assembly or packaging of the implant 100. In that case, the surgeon or other user would receive the implant 100 with the base material already contained within the implant 100. In an example, the surgeon or other user can insert the bone cement into the implant 100 during or prior to the procedure to insert the implant 100 into the joint space. The bone cement can be supplied with the implant 100 or separately. The surgeon or other user can also inject additional antibiotics into the implant 100 based on a particular patient's infection and circumstances.

An example of bone cement usable with the implant 100 includes, but is not limited to, acrylic bone cements containing components such as polymethyl methacrylate (PMMA), poly(methyl methacrylate-co-styrene) (PMMA/Styrene), poly(methyl methacrylate-co-methyl acrylate) (PMMA/MA). Also usable with the implant 100 includes, but not limited to, inorganic bone cements containing components such as calcium, magnesium, hydroxyapatite, phosphates, sulfates and silicates. Examples of antibiotics usable with the implant 100 include, but are not limited to, penicillin, erythromycin, colistin, cephalorines, polymyxin, gentamicin, tobramycin, and vancomycin, which are soluble in an aqueous solution. Examples of antimicrobials usable with the implant 100 includes, but is not limited to: silver and silver ions. An elution or release rate of the antibiotic from the bone cement (and through the perforations 104) can depend in part on a porosity of the bone cement, which can be controlled, in part, during mixing and curing of the bone cement. There may not be consistency within the same implant or from implant to implant in the porosity of the bone cement, and thus the elution or release rate of the antibiotic through the perforations 104 can vary.

FIG. 5 shows the implant 100 of FIG. 3 and another example of a filler device 130. In the example of FIG. 5, the base material is an antimicrobial infused material and the filler device 130 includes a closeable port for inserting the material into the implant 100 prior to implantation of the implant 100. The base material can be compressed into filler device 130. Upon decompression of the material, the material can flow from the port 130 and into the interior of the implant 100. As described above in reference to FIG. 4, the implant 100 can include the feature 124 which receives or attaches to the filler device 130.

The antimicrobial infused material can be any known material usable in combination with an antimicrobial such that the material can be inserted into the interior of the implant and the antimicrobial can be released through the perforations 104 in the exterior shell 102 of the implant 100. The material can include, but is not limited to, gels, foams, fibers, woven and non-woven materials, any of which can be made of synthetic or natural polymers. Although some of these types of materials may not be flowable, at least some may be compressible. As such, the compressible material can be compressed into a syringe or other device and then released into the interior of the implant 100. A porosity of these materials can be more uniform as compared to bone cement. An elution or release rate of the antimicrobials from these materials can be controlled, in part, by varying the pore size in nonresorbable materials or by the resorption rate of the polymer in resorbable mateials. Synthetic biodegradable polymers such as poly-(lactide-co-glycolide)copolymers, polycaprolactone, polyanhydrides, polyhydroxybutyrate-co-hydroxyvalaerate, polyhydroxyalkanoates can be used for antibiotic elution or release. Natural polymers such as collagen chitosan and methylcellulose can also be used for antibiotic elution or release.

As stated above, the material containing the antibiotic or antimicrobial can be inserted into the interior of the implant 100 prior to implantation of the implant 100 in the body. The implant 100 can be provided to the surgeon or other user with the material already contained within the implant 100. (As mentioned above, shrink wrap or something similar can be used to contain the material inside the implant 100 until implantation.) Alternatively, the implant and the material containing the antibiotic or antimicrobial can be separately provided to the surgeon or other user and the material can be injected into the implant 100 during preparation of the implant 100 for implantation.

In an example in which a foam is used to temporarily contain the antibiotic or antimicrobial, additional antibiotic or antimicrobial can be injected into the foam after the original antibiotic or antimicrobial has been released. This can be beneficial if the infection has not been eradicated. In an example, the foam or other similar type material can be degradable or resorbable. In that case, another material can be injected into the implant 100; for example, bone cement can be injected into the implant 100 to provide additional structural integrity to the implant 100, particularly if the implant 100 is a permanent implant. In an example, the foam, gel or other material can be inserted into the implant 100 prior to the surgeon or other user receiving the implant. In that case, the antibiotic or antimicrobial can already be contained within the material, or alternatively, the surgeon or other user can inject the antibiotic or antimicrobial as part of the surgical procedure for inserting the implant 100. In an example, the implant 100 and the foam, gel or other material can be separate and the surgeon or other user can insert the foam, gel or other material into the implant as part of the surgical procedure; the antibiotic or antimicrobial can be already injected into the foam, gel or other material, or this step can also be performed by the surgeon or other user.

FIG. 6 shows an example of an implant 200 which can be similar to the implant 100 but can also include a femoral head 254. In an example, the femoral head 254 can fit over an extension (not shown) extending from a body 256 of the implant 200. The femoral head 254 can be made of the same or a different material than an exterior shell 202 of the implant 200. As shown in FIG. 6, the femoral head 254, or a portion thereof, can include perforations 260 formed in an exterior shell 262 of the femoral head 254, which can be similar to perforations 204 in the exterior shell 262. Similar to the body 256, the femoral head 254 can also be filled or partially filled with an antibiotic or antimicrobial containing material prior to implantation of the implant 200. Any of the materials described above can be used and can be inserted into the femoral head 254 using any of the methods described above.

The femoral head 254 can include a cap 264 configured to cover a portion of the exterior shell 262 of the femoral head 254. The cap 264 can be configured to act as an articulating surface for articulation with an acetabular shell (see FIG. 2.) The cap 264 can cover more or less of the exterior shell 262, relative to what is shown in FIG. 6. In other examples, the implant 200 can exclude the cap 264 and the exterior shell 262 of the femoral head 254 can act as an articulating surface with the acetabular shell. The cap 264 can be formed from metal or other known materials suitable for an articulation surface. In an example, the cap 264 can be formed from titanium.

In an example, essentially all of the femoral head 254 can include perorations in the exterior shell 262. The cap 264 can be sized to fit over a portion of the femoral head 254. The antibiotic or antimicrobial can flow through the perforations 260 in an area of the exterior shell 262 covered by the cap 264.

The base material containing the antibiotic or antimicrobial can be inserted into the interior of the implant 200 using the methods and devices described above in reference to the implant 100.

A method of making an implant that is similar to the implants 100 and 200 described herein can include forming an exterior shell having an overall shape that defines a shape of the implant. The exterior shell can be formed, for example, out of metal. A plurality of perforations can be formed in at least a part of the exterior shell. The perforations can be formed before or after the metal is shaped to form the exterior shell. In an example, the exterior shell is formed around a core material. The method can include attaching a femoral head to the exterior shell of the implant. The femoral head can be formed similarly to the rest of the implant and can include perforations.

As described above, the implants 100 and 200 can be designed as temporary or permanent revision implants. The implants 100 and 200 can be filled with an antibiotic or antimicrobial containing material prior to implantation and the antibiotic or antimicrobial can flow out of the implant 100 or 200 after implantation in order treat the infection. The implants 100 and 200 can also be designed such that additional materials can be inserted into the interior of the implant 100 or 200 after the implant 100 or 200 has been implanted.

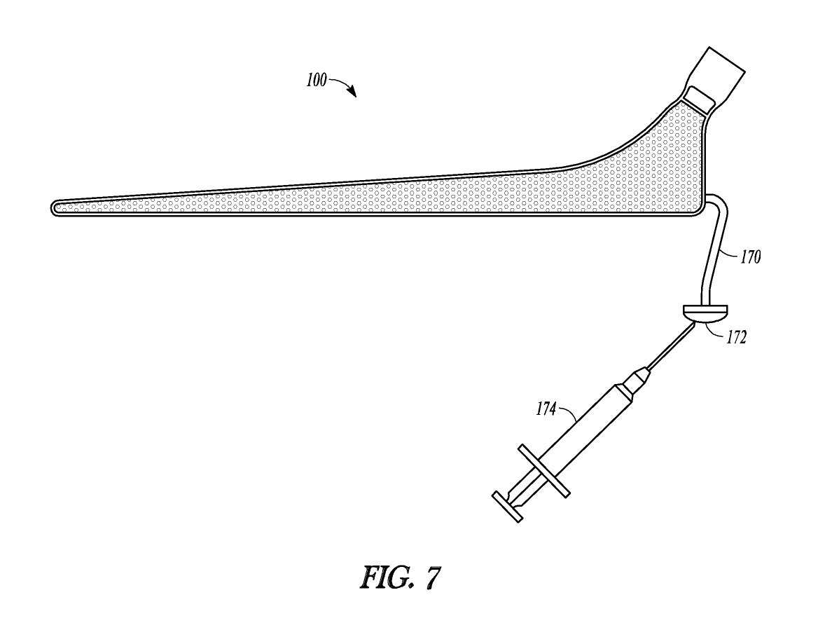

As shown in FIG. 7, in an example, the implant 100 can be fitted with an implantable tube 170 attached to a septum port 172. In an example, the port 172 can be a sub-dermal port and implanted with the implant 100 or can be implanted in a subsequent surgery after the implantation of the implant 100. The port 172 can be used to store and deliver additional antimicrobials or antibiotics into the interior of the implant 100 to further treat the infection. FIG. 7 shows an example of a delivery device 174 for filling the port 172. Other types of delivery devices can be used to fill the port 172.

By initially filling the implant 100 with a base material and designing the implant 100 such that it can receive additional materials, for example, by injection with a hypodermic needle through a sub-dermal septum port, additional or different antibiotics or antimicrobials can be added without requiring an additional surgery. Through use of a sub-dermal port, the patient can be mobile during the treatment of the infection. Moreover, other types of materials can be added using the port 172. In an example, the implant 100 and the port 172 can first be filled with resorbable materials. An agent can also be used to delay bone ingrowth until the infection is eradicated. Samples of the fluids surrounding the implant 100 can be monitored and tested to determine, for example, if and when the infection is eradicated.

In an example, if it is determined that the infection is eradicated, a solution can be inserted into the port 172 to irrigate the implant 100 and the area surrounding the implant 100. A scaffold material or an osteoinductive agent can then be supplied to the implant 100 to induce bone ingrowth into the implant 100. In another example, the implant 100 can be injected with a structural material, such as, for example, bone cement, to provide additional mechanical strength to the implant 100 and to permanently fix the implant 100 to the bone through the perforations 104 in the implant.

The implants described herein can include patient specific implants that can be custom made for a particular patient. The patient specific implant can be designed, for example, from a 3D model based on a scan or other imaging of that particular patient's bone joint. Although the implants 100 and 200 are hip prostheses, it is recognized that other types of implants can be formed as described herein and used for treating an infection on or around a joint space, including, but not limited to, knee implants, shoulder implants and elbow implants. Delivery of the base material into or out of the other types of implants can be similar to as described herein in reference to the hip implants 100 and 200.

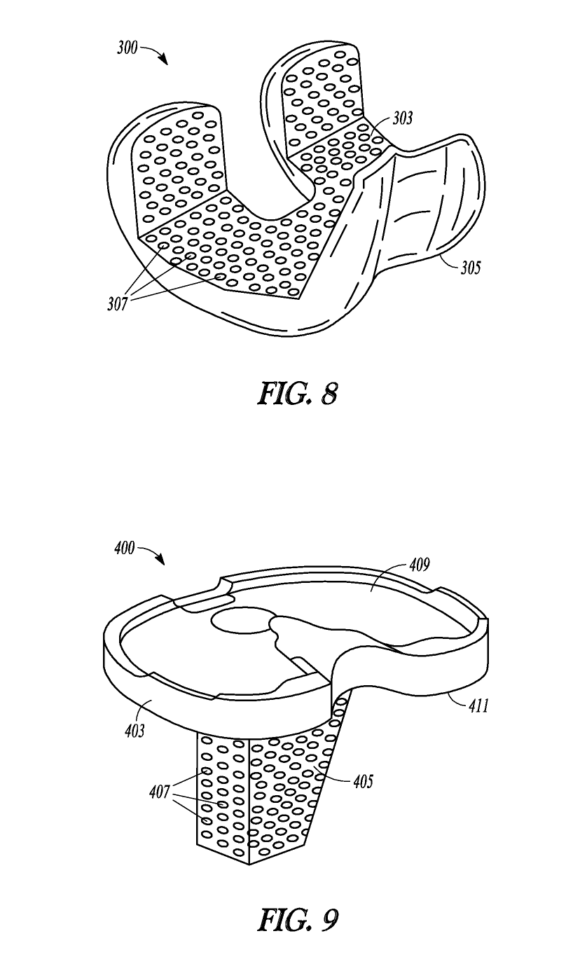

FIGS. 8 and 9 illustrate examples of knee implants for treating an infection, as described below. FIG. 8 shows an example of a femoral implant 300, which is configured to be attached to a distal end of a femur. The femoral implant 300 can include a bone contacting surface 303 and an articulating surface 305. As shown in FIG. 8, all or a portion of the bone contacting surface 303 can include a plurality of perforations 307 that can be used to release an antibiotic or antimicrobial from an interior of the femoral implant 300.

FIG. 9 shows an example of a tibial implant 400, which is configured to be attached to a proximal end of a tibia and can include a base portion 403 and a keel 405. The keel 405 can be configured to extend into an intramedullary canal of the tibia and can improve fixation of the tibial implant 400 to the tibia. As shown in FIG. 9, all or a portion of the keel 405 can include a plurality of perforations 407 for releasing an antimicrobial from an interior of the tibial implant 400. The base 403 of the tibial implant 400 can include a superior surface 409 and an inferior surface 411 opposite the superior surface 409. The superior surface 409 can be configured for engagement with a bearing component designed for interaction with a femoral component like the femoral implant 300. The inferior surface 411 can be a bone contacting surface that contacts the resected surface of the tibia when the tibial implant 400 is attached to the tibia. Although not shown in FIG. 9, all or a portion of the inferior surface 411 can include perforations similar to the perforations 407.

The femoral 300 and tibial 400 implants can be configured similar to the hip implants 100 and 200 described above to release an antibiotic or antimicrobial. Any of the materials described above can be used and can be inserted into the implants 300 and 400 using any of the methods described above. The implants 300 and 400 can be used as temporary or permanent revision implants.

It is recognized that there are other designs of femoral and tibial implants, which can incorporate the designs described herein for treating an infection. For example, other types of securement or fixation structures can be used for the tibial implant 400 in addition to or in place of the keel 405, such as, for example, one or more pegs. These other types of fixation structures can also include perforations similar to the perforations 407 shown on the keel 405.

As described above, any of the implants 100, 200, 300 or 400 can be used as part of a two-stage revision procedure in which the implant 100, 200, 300 or 400 is used as a temporary spacer to treat an infection or alternatively, the implants 100, 200, 300 or 400 can be used as a permanent revision implant that can be left inside the body, yet simultaneously treat the infection given the release of an antibiotic or antimicrobial. In examples in which the implant is configured as a permanent revision implant, the implant can include additional materials on an exterior of the implant that can be used to aid in permanent placement of the implant. These additional materials can be used in combination with the perforations on the exterior shell of the implant, as described above and shown in the figures. In an example, these additional materials are porous materials, such as for example, fiber metal or porous tantalum, and can include channels or a perforated substrate, such that the antibiotic or antimicrobial can flow through the perforations on the exterior shell and through these additional materials. Another example includes porous hydroxyapatite or other types of materials that can initially release the antibiotic or antimicrobial to an area surrounding the implant and then can promote bone ingrowth. In an example, a hydroxyapatite coating can be soaked into the implant just prior to insertion of the implant.

A method of treating an infection on or around a joint space can include providing an implant similar to the implants 100 and 200 described herein and providing a base material as described herein. The base material can include an antibiotic or antimicrobial and can be inserted into the interior portion of the implant. The method can include inserting the implant in the joint space such that the perforations in the exterior shell release the antimicrobial or antibiotic in the base material from the interior portion of the implant. In an example, the method can include attaching a port to the implant for delivering additional materials to the interior portion of the implant. The port can be implanted inside the body at the same time as the implant or at a later date. Following the surgery for implantation of the implant and port, the port can be filled with a hypodermic needle, or a similar device, without having to open up the body of the patient in a subsequent surgery. The additional materials in the port can be selected based, in part, on the needs of the patient and whether the infection has been eliminated, as well as whether the implant is intended to remain inside the body as the permanent revision implant. The additional materials, as described above, can include, but are not limited to, an irrigation material, a scaffold material, and an osteoinductive agent.

The implants and methods described herein, including the components usable with the implant (such as the filler device and ports), can be used to effectively treat an infection in the body near the original implant. The implant can be used as a permanent revision implant and the ability to deliver additional materials to the implant, after the revision surgery, allows for flexibility and increased effectiveness of the implant. The implants and methods described herein can avoid having two separate surgeries—the first being to implant the device for treating the infection; the second to replace the temporary implant with the permanent revision implant. Moreover, the method for making the implant is simplified as compared to known methods, which can require the use of molds. The mechanical strength provided by the exterior shell of the implant makes the implant suitable for use as a permanent revision implant. The implant can be configured with articulation surfaces that can have improved properties over existing implants used for treating infections.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An implant comprising:
an exterior shell having an overall shape that defines a shape of the implant; and
an interior portion, at least a part of the interior portion being hollow and configured to contain a base material including an antimicrobial or antibiotic, wherein the exterior shell includes a plurality of perforations formed in at least a part of the exterior shell, and the perforations are configured to release the antimicrobial or antibiotic in the base material from the interior portion of the implant.

2. The implant of claim 1, further comprising a core material located within the interior portion.

3. The implant of claim 2, wherein the core material is formed of a metal or metal alloy.

4. The implant of claim 3, wherein the core material is cobalt chrome.

5. The implant of claim 1, wherein the exterior shell is formed of a metal or metal alloy.

6. The implant of claim 5, wherein the exterior shell is formed of at least one of titanium, stainless steel, cobalt chrome, or combinations thereof.

7. The implant of claim 1, further comprising a feature on the exterior shell or attachable to the exterior shell, wherein the feature is configured to facilitate injection of the base material into the interior portion of the implant.

8. The implant of claim 1, wherein the base material is a bone cement, gel, foam or fiber.

9. The implant of claim 1, wherein the implant is configured for use as a permanent revision implant.

10. A system for treating an infection on or around a joint space, the system comprising:
an implant comprising:
an exterior shell having an overall shape that defines a shape of the implant and having a plurality of perforations formed in at least a part of the exterior shell; and
an interior portion, at least a part of the interior portion being hollow;
a base material including an antibiotic or antimicrobial; and
a filler device configured for releasable attachment to the implant to deliver the base material into the interior portion of the implant, wherein the interior portion is configured to contain the base material, and the perforations are configured to release the antimicrobial or antibiotic in the base material from the interior portion of the implant.

11. The system of claim 10, the implant further comprising a core material located within the interior portion of the implant.

12. The system of claim 11, wherein the base material is releasably contained between an interior surface of the shell and the core material.

13. The system of claim 10, wherein the implant includes an articulation surface.

14. The system of claim 13, wherein the articulation surface is a cap that is inserted over a part of the exterior shell.

15. The system of claim 10 further comprising:
a film configured to wrap around the implant to secure the implant and immobilize the base material within the interior portion of the implant, wherein the film is removable prior to inserting the implant inside a body of a patient.

16. The system of claim 10, wherein the film is a shrink wrap.

17. The system of claim 10 further comprising:
a port configured for connection to the implant and configured to deliver a second base material into the interior of the implant.

18. The system of claim 17 wherein the port is a sub-dermal port configured to be implanted into a body of a patient at the same time as the implant.

19. The system of claim 10, wherein the implant is a hip implant.

20. The system of claim 10, wherein the implant is a knee implant.

21. The system of claim 10, wherein the base material is a bone cement, gel, foam or fiber.
